(19) United States
(12) Patent Application Publication
(10) Pub. No.: US 2024/0293147 A1
(43) Pub. Date: Sep. 5, 2024

(54) SHUNTING CATHETERS

(71) Applicant: THERAHEART INC., Irvine, CA (US)

(72) Inventor: Rani Abdullah Mahmoudi, Huntington Beach, CA (US)

(21) Appl. No.: 18/593,832

(22) Filed: Mar. 1, 2024

Related U.S. Application Data
(60) Provisional application No. 63/449,450, filed on Mar. 2, 2023.

(51) Int. Cl. A61B 17/34; A61B 17/00; A61B 34/20; A61B 90/00

(52) U.S. Cl. CPC A61B 17/3403; A61B 17/00234; A61B 34/20; A61B 90/39; A61B 2017/00199; A61B 2017/00252; A61B 2017/00305; A61B 2017/00411; A61B 2017/00544; A61B 2017/00557; A61B 2034/2046; A61B 2090/3966

(57) ABSTRACT

At least some embodiments of the present disclosure are directed to systems and methods for creating a shunt in a patient. In some embodiments, a shunting catheter includes a catheter shaft having a distal end and a proximal end, the catheter shaft including a shaft lumen; a shunting element disposed in the shaft lumen at a first state and extended from the catheter shaft at a second state; and an apposition element disposed proximate to the shunting element, the apposition element being protruded from the catheter shaft at the second state.

### CROSS REFERENCE TO RELATED APPLICATIONS

This application claims priority to U.S. Provisional Application No. 63/449,450, filed Mar. 2, 2023, incorporated by reference herein for all purposes.

### FIELD

Certain embodiments of the present disclosure relate to medical systems, apparatus, and methods for creating a shunt in a patient. More specifically, some embodiments of the present disclosure relate to medical systems, apparatus, and methods for creating a shunt on a cardiovascular system wall in a patient.

### BACKGROUND

Heart failure is a serious condition that happens when heart cannot pump enough blood and oxygen to support other organs in your body. Heart failure is classified according to left ventricular (LV) function as “heart failure with reduced ejection fraction (EF)” (HFrEF; EF<40%), “midrange EF” (HFmrEF; EF 40-49%), or “preserved EF” (HFpEF; EF≥50%). About half the patients with heart failure have HFpEF. HFpEF generally happens when LV and left atrial filling pressures increase significantly during exercise, with an associated increase in pulmonary pressures leading to pulmonary congestion. Structural interventions to lower elevated either left or right atrial filling pressures are gaining attention.

Studies in heart failure show that lowering left atrial pressure may reduce cardiovascular events while improving functional capacity. The creation of an interatrial shunt has emerged as a therapy to decompress the left atrium in patients with acute and chronic left HF. As such, attention has turned toward the development of interatrial shunt devices (IASDs) as a means of reducing the detrimental increase in left-sided filling pressures with exercise in an effort to improve symptomatology. The IASDs may be used to treat various kinds of heart failure and/or other diseases that may result in too high of a pressure in the right atrium of a patient.

### SUMMARY

Current IASDs reside in the interatrial septum, with risk for right-to-left shunting and systemic embolization. Moreover, preservation of the interatrial septum is important with an increasing number of left-sided transseptal transcatheter interventions. Ways to improve IASDs for safer and better procedures are needed.

According to some embodiments of the present disclosure, a shunting catheter includes: a catheter shaft having a distal end and a proximal end, the catheter shaft including a shaft lumen; a shunting element disposed in the shaft lumen at a first state and extended from the catheter shaft at a second state; and an apposition element disposed proximate to the shunting element, the apposition element being protruded from the catheter shaft at the second state.

In certain embodiments, the catheter shaft defines a first axis; wherein the shunting element defines a second axis at the second state; wherein the second axis and the first axis form an angle greater than zero degree. In some embodiments, the shunting element includes an expandable element that is expanded at the second state. In certain embodiments, the shunting element includes a tube to support the expandable element. In some embodiments, the tube includes a plurality of cuts generally perpendicular to the second axis.

In some embodiments, the catheter shaft has a shaft opening, wherein the shunting element extends from the catheter shaft through the shaft opening. In certain embodiments, the shaft opening is not at the distal end of the catheter shaft. In some embodiments, a portion of the catheter shaft between the shaft opening and the distal end has a curve. In some embodiments, the shunting element includes a guidewire. In certain embodiments, the shunting element includes a puncture element.

In certain embodiments, the shunting catheter further includes: an outer shaft disposed outside of at least a part of the catheter shaft; wherein the apposition element is disposed within the outer shaft. In some embodiments, the apposition element includes a braided element protruded from the catheter shaft at the second state. In certain embodiments, the braided element includes one or more nickel titanium wires.

According to some embodiments, a shunting catheter includes a catheter shaft having a distal end and a proximal end, the catheter shaft including a shaft lumen; a shunting element disposed in the shaft lumen at a first state and extended from the catheter shaft at a second state; wherein the catheter shaft defines a first axis; wherein the shunting element defines a second axis at the second state; wherein the second axis and the first axis form an angle greater than zero degree. In some embodiments, the distal end of the catheter shaft has a curve.

In certain embodiments, the shunting catheter further includes an apposition element disposed proximate to the shunting element, the apposition element being protruded from the catheter shaft at the second state.

According to some embodiments, a shunting catheter system includes a shunting catheter including: a catheter shaft having a distal end and a proximal end, the catheter shaft including a shaft lumen; a shunting element disposed in the shaft lumen at a first state and extended from the catheter shaft at a second state; and an apposition element disposed proximate to the shunting element, the apposition element being protruded from the catheter shaft at the second state. In some embodiments, the shunting catheter system further includes an energy source connected to the shunting catheter; and a controller connected to the energy source including one or more processors; wherein the one or more processors are configured to control the energy source to deliver energy to the shunting catheter.

In certain embodiments, the shunting catheter system further includes an imaging device including: one or more visualization elements disposed proximate the shunting element for determining a location of the shunting element within a heart of a patient, and a display for visualizing the location.

According to certain embodiments, a method for creating a shunt, includes deploying a shunting catheter in a first state, the shunting catheter including: a catheter shaft having a distal end and a proximal end, the catheter shaft including a shaft lumen; a shunting element having a proximal end and a distal end, wherein the shunting element is disposed in the shaft lumen at the first state; and a puncture element disposed proximate to the distal end of the shunting element; disposing the shunting catheter approximate to a target location of a patient; operating the shunting catheter to a second state, wherein the shunting element extends from the catheter shaft at an angle greater than zero degree at the proximal end of the shunting element at the second state; puncturing, using the puncture element, an opening at the target location; and expanding the opening using the shunting element.

In some embodiments, the shunting catheter further includes an apposition element disposed proximate to the shunting element, wherein the apposition element is protruded from the catheter shaft at the second state. In certain embodiments, the catheter shaft has a shaft opening, wherein the shunting element extends from the catheter shaft through the shaft opening.

In some embodiments, the method further includes determining a location of the shunting element using an imaging device. In certain embodiments, the imaging device includes one or more visualization elements disposed proximate the shunting element. In some embodiments, the shunting element includes an expandable element that is expanded at the second state.

In certain embodiments, the method further includes treating tissue surrounding the opening using the shunting element. In some embodiments, the target location is at a coronary sinus of the patient. In certain embodiments, deploying the shunting catheter in the first state includes inserting the shunting catheter through a superior vena cava or an inferior vena cava of the patient into a coronary sinus of the patient.

In some embodiments, the method further includes removing the shunting catheter from the patient. In certain embodiments, the method further includes generating the shunt using the shunting element; wherein the shunt includes an expanded opening between the coronary sinus and a left atrium of the patient. In some embodiments, the shunt does not include any implant.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

FIG. 1 is a diagram illustrating an exemplary clinical setting for treating a heart of the patient, using a shunting catheter system, in accordance with embodiments of the present disclosure.

FIG. 2 is a schematic diagram illustrating an example of a shunting device to be deployed in a heart of a patient, in accordance with embodiments of the present disclosure.

FIG. 3 is a schematic diagram of a side view of an example of a shunting device and a perspective view of an apposition element of the shunting device, in accordance with embodiments of the present disclosure.

FIG. 4 is schematic diagrams of a cross-sectional view of an example of a shunting catheter, in accordance with embodiments of the present disclosure.

FIGS. 5A-5B are schematic diagrams of a cross-sectional view of an example of a shunting catheter, in accordance with embodiments of the present disclosure.

FIGS. 6A-6C are schematic diagrams of a side view of an example of a shunting catheter, in accordance with embodiments of the present disclosure.

FIGS. 7A-7B are schematic diagrams of an example of a shunting catheter, in accordance with embodiments of the present disclosure.

FIG. 8 is a flow diagram illustrating a process of creating a shunt in a patient, in accordance with embodiments of the present disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

The following detailed description is exemplary in nature and is not intended to limit the scope, applicability, or configuration of the invention in any way.

Rather, the following description provides some practical illustrations for implementing exemplary embodiments of the present invention. Examples of constructions, materials, and/or dimensions are provided for selected elements. Those skilled in the art will recognize that many of the noted examples have a variety of suitable alternatives.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein. The use of numerical ranges by endpoints includes all numbers within that range (e.g., 1 to 5 includes 1, 1.5, 2, 2.75, 3, 3.80, 4, and 5) and any number within that range.

Although illustrative methods may be represented by one or more drawings (e.g., flow diagrams, communication flows, etc.), the drawings should not be interpreted as implying any requirement of, or particular order among or between, various steps disclosed herein. However, some embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items and, similarly, a subset or subgroup of items may include one or more items. A “plurality” means more than one.

As used herein, the term “based on” is not meant to be restrictive, but rather indicates that a determination, identification, prediction, calculation, and/or the like, is performed by using, at least, the term following “based on” as an input. For example, predicting an outcome based on a particular piece of information may additionally, or alternatively, base the same determination on another piece of information. In some embodiments, the term “receive” or “receiving” means obtaining from a data repository (e.g., database), from another system or service, from another software, or from another software component in a same software. In certain embodiments, the term “access” or “accessing” means retrieving data or information, and/or generating data or information.

There are various approaches for creating an interatrial shunt, which is a connection or gateway between the left and right atria of a patient's heart for blood to flow through. In some embodiments, examples of interatrial shunt devices (IASDs) include implants or shunting catheters. For example, devices reside in the interatrial septum, with risk for right-to-left shunting and systemic embolization. In some examples, preservation of the interatrial septum is important with an increasing number of left-sided transseptal transcatheter interventions. Ways to improve IASDs for safer and better procedures are needed. At least some embodiments of the present disclosure are directed to a shunting catheter for deployment through a patient's coronary sinus (CS) for creating a shunt between the CS and the patient's left atrium (LA). At least some embodiments of the present disclosure are directed to a shunting catheter for deployment through a patient's atrial septum (AS) for atrial septal shunting.

A patient's CS ostium may have a diameter of from about 10 mm to about 20 mm. As the CS is a relatively small vessel, at least some embodiments of the present disclosure are directed to features of a shunting catheter that helps protect a patient's vessels during deployment and/or elements for stabilizing the catheter during the procedure. In embodiments, the shunting catheter includes a catheter shaft, a shunting element, and an apposition element disposed proximate to the shunting element. In some embodiments, the catheter shaft is made of flexible materials that bends according to the anatomy of the CS to conform to the shape of the patient's CS. In yet some embodiments, the catheter shaft includes a stabilizing element such as distal tip that has a curve (e.g., a pre-existing curve) conforming to the shape of a patient's CS to help stabilize the catheter and minimize potential damage to a patient's tissue wall (e.g., the vessel wall of a patient's CS).

In some embodiments, the apposition element is protruded from the catheter shaft during deployment to help stabilize the catheter at a desired location for creating the shunt. In certain embodiments, the shunting element further includes an expandable element (e.g., a balloon) and a tube (e.g., a hypotube) to support the expandable element. The tube may have a plurality of cuts along the tube to help facilitate bending of the tube. In some embodiments, a shunt is formed in a patient's CS vessel by creating an opening between the patient's CS and LA. In certain embodiments, the shunting catheter is inserted through the patient's superior vena cava (SVC) via a transjugular approach. In certain embodiments, the shunting catheter is inserted through the patient's inferior vena cava (IVC) via a transfemoral approach.

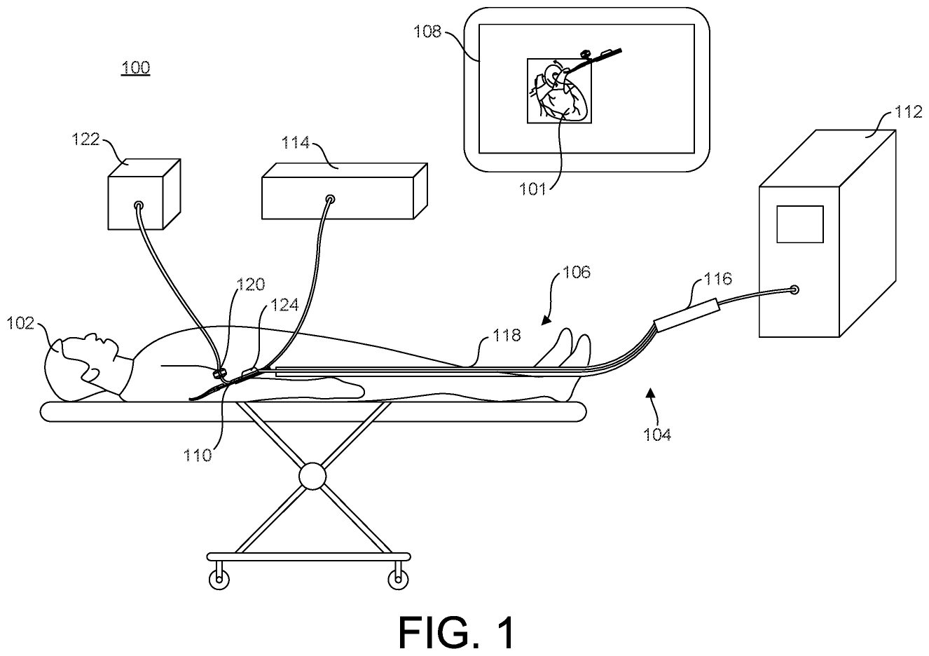

FIG. 1 is a diagram illustrating an exemplary clinical setting 100 for treating a heart 101 of the patient 102, using a shunting catheter system 104, in accordance with embodiments of the present disclosure. The shunting catheter system 104 includes a shunting device 106. As will be appreciated by the skilled artisan, the clinical setting 100 may have other components and arrangements of components that are not shown in FIG. 1. In some embodiments, the shunting catheter system 104 includes or is coupled to an imaging system (e.g., an X-ray system) which may include one or more visualization elements and a display 108. In some embodiments, one or more visualization elements may be disposed on the shunting device 106. In certain embodiments, the imaging system can help guide a physician's operation of the shunting catheter 110 during procedure.

The shunting device 106 includes a shunting catheter 110, a controller 112, and an energy source 114 (e.g., a generator). The controller 112 is configured to control functional aspects of the shunting device 106. In embodiments, the controller 112 is configured to control the energy source 114 to deliver energy to the shunting catheter 110. The controller 112 may be connected to the one or more visualization elements to facilitate positioning of the shunting catheter 110 in a patient's heart during procedure. In some embodiments, the energy source 114 is connected to the controller 112. In yet some embodiments, the energy source 114 may be incorporated into the controller 112.

As will be appreciated by the skilled artisan, the depiction of the shunting catheter system 104 shown in FIG. 1 is intended to provide a general overview of the various components of the shunting catheter system 104 and is not in any way intended to imply that the disclosure is limited to any set of components or arrangement of the components. For example, the skilled artisan will readily recognize that additional hardware components, e.g., breakout boxes, workstations, and the like, can and likely will be included in the shunting catheter system 104.

According to some embodiments, the shunting device 106 includes a handle 116, a catheter shaft 118, a puncture element (e.g., a puncture needle) configured to puncture through a tissue wall, and a shunting element 120 configured to provide shunting at a target location. In certain embodiments, the shunting element 120 is inflatable and connected to an inflation source 122. In some instances, the shunting element 120 includes an expandable element (e.g., a balloon). In certain embodiments, the shunting element 120 is connected to the energy source 114 to provide shunting. For example, the shunting element 120 includes electrodes to receive electrical power from the energy source 114 to deliver ablation energy to the target location (e.g., a target tissue) at a cardiovascular system (e.g., a circulatory system) wall. In certain embodiments, the handle 116 is configured to be operated by a user to position the puncture element and the shunting element 120 at the desired anatomical location. The catheter shaft 118 generally defines a longitudinal axis of the shunting catheter 110. In some embodiments, the shunting element 120 may be connected to a shunting element shaft positioned within the catheter shaft 118 at a first state (e.g., before a deployment and/or during a deployment to position the shunting element 120). In certain embodiments, the shunting element shaft has a pre-determined curve. In some examples, the shunting element shaft has a pre-determined curve for the shunting element to deploy. In certain embodiments, the shunting element shaft is extended from the catheter shaft 118 at a second state (e.g., a shunting state to use the shunting element).

According to certain embodiments, during deployment, the shunting device 106 including the catheter shaft 118 enters through a patient's CS ostium located in the patient's right atrium. The shunting device 106 may then be oriented through one or more mechanisms in the patient's CS, as will be discussed in more details below. In some embodiments, in order to conform to the shape of the patient's CS, the catheter shaft 118 is made of flexible materials that may bend according to the anatomy of the CS.

In certain embodiments, the shunting catheter 110 includes an apposition element 124 disposed proximate to the shunting element 120. In some embodiments, the apposition element is disposed within a shaft (e.g., an outer shaft) at the first state. In some embodiments, the apposition element 124 is protruded from the catheter shaft 118 at the first state and/or at the second state. In certain embodiments, the apposition element 124 can appose to a cardiovascular system wall (e.g., the front wall or back wall of the CS, a left atrium wall, a right atrium wall, etc.) at the second state, for example, to help position and/or stabilize the shunting element 120. In certain embodiments, the apposition element 124 includes a braid structure. In some embodiment, the apposition element 124 may include a nitinol braid that can be held within the catheter shaft 118.

According to some embodiments, various components (e.g., the controller 112) of the shunting catheter system 104 may be implemented on one or more computing devices. A computing device may include any type of computing device suitable for implementing embodiments of the disclosure. Examples of computing devices include specialized computing devices or general-purpose computing devices such as workstations, servers, laptops, portable devices, desktop, tablet computers, hand-held devices, general-purpose graphics processing units (GPGPUs), and the like, all of which are contemplated within the scope of FIG. 1 with reference to various components of the shunting catheter system 104.

In some embodiments, a computing device (e.g., the controller 112) includes a bus that, directly and/or indirectly, couples the following devices: a processor, a memory, an input/output (I/O) port, an I/O component, and a power supply. Any number of additional components, different components, and/or combinations of components may also be included in the computing device. The bus represents what may be one or more busses (such as, for example, an address bus, data bus, or combination thereof). Similarly, in some embodiments, the computing device may include a number of processors, a number of memory components, a number of I/O ports, a number of I/O components, and/or a number of power supplies. Additionally, any number of these components, or combinations thereof, may be distributed and/or duplicated across a number of computing devices. In some embodiments, various components or parts of components (e.g., controller 112, shunting catheter 110, etc.) can be integrated into a physical device.

In some embodiments, the shunting catheter system 104 includes one or more memories (not illustrated). The one or more memories includes computer-readable media in the form of volatile and/or nonvolatile memory, transitory and/or non-transitory storage media and may be removable, nonremovable, or a combination thereof. Media examples include Random Access Memory (RAM); Read Only Memory (ROM); Electronically Erasable Programmable Read Only Memory (EEPROM); flash memory; optical or holographic media; magnetic cassettes, magnetic tape, magnetic disk storage or other magnetic storage devices; data transmissions; and/or any other medium that can be used to store information and can be accessed by a computing device such as, for example, quantum state memory, and/or the like. In some embodiments, the one or more memories store computer-executable instructions for causing a processor (e.g., the controller 90) to implement aspects of embodiments of system components discussed herein and/or to perform aspects of embodiments of methods and procedures discussed herein.

Computer-executable instructions may include, for example, computer code, machine-useable instructions, and the like such as, for example, program components capable of being executed by one or more processors associated with a computing device. Program components may be programmed using any number of different programming environments, including various languages, development kits, frameworks, and/or the like. Some or all of the functionality contemplated herein may also, or alternatively, be implemented in hardware and/or firmware.

In some embodiments, the memory may include a data repository which may be implemented using any one of the configurations described below. A data repository may include random access memories, flat files, XML files, and/or one or more database management systems (DBMS) executing on one or more database servers or a data center. A database management system may be a relational (RDBMS), hierarchical (HDBMS), multidimensional (MDBMS), object oriented (ODBMS or OODBMS) or object relational (ORDBMS) database management system, and the like. The data repository may be, for example, a single relational database. In some cases, the data repository may include a plurality of databases that can exchange and aggregate data by a data integration process or software application. In an exemplary embodiment, at least part of the data repository may be hosted in a cloud data center. In some cases, a data repository may be hosted on a single computer, a server, a storage device, a cloud server, or the like. In some other cases, a data repository may be hosted on a series of networked computers, servers, or devices. In some cases, a data repository may be hosted on tiers of data storage devices including local, regional, and central.

Various components of the shunting catheter system 104 can communicate via or be coupled to via a communication interface, for example, a wired or wireless interface. The communication interface includes, but is not limited to, any wired or wireless short-range and long-range communication interfaces. The wired interface can use cables, umbilicals, and the like. The short-range communication interfaces may be, for example, local area network (LAN), interfaces conforming to known communications standards, such as Bluetooth™ standard, IEEE 802 standards (e.g., IEEE 802.11), a ZigBee™ or similar specification, such as those based on the IEEE 802.15.4 standard, or other public or proprietary wireless protocol. The long-range communication interfaces may be, for example, wide area network (WAN), cellular network interfaces, satellite communication interfaces, etc. The communication interface may be either within a private computer network, such as intranet, or on a public computer network, such as the internet. Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

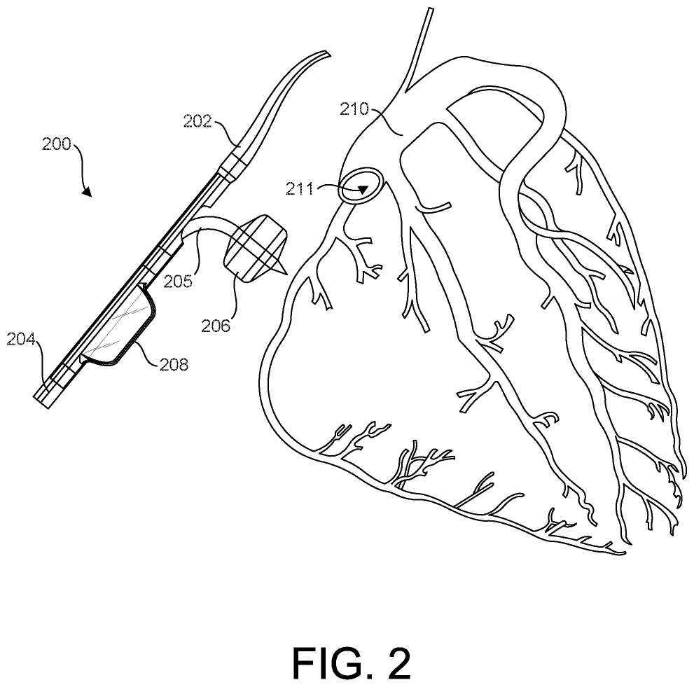

FIG. 2 is a schematic diagram illustrating an example of a shunting device 200 to be deployed in a heart of a patient, in accordance with embodiments of the present disclosure. FIG. 2 is merely an example. One of the ordinary skilled in the art would recognize many variations, alternatives, and modifications. As shown, the shunting device 200 includes a shunting catheter 202 to be delivered through a patient's coronary sinus (CS) 210 via the CS ostium 211. In some embodiments, the shunting catheter 202 includes a catheter shaft 204, a shunting element 206, and an apposition element 208. In certain embodiments, the catheter shaft 204 has a curve at its distal end 205. In some embodiments, as illustrated, the shunting element 206 is extended from the catheter shaft 204 at a second state (e.g., a state to provide shunting). In certain examples, the shunting element 206 forms an angle greater than 30 degrees From the distal end 205 of the catheter shaft 204. In some embodiments, the shunting element 206 forms an angle proximate to 90 degrees From the catheter shaft 204. In some embodiments, the shunting element 206 forms an angle in the range of 60 degrees to 120 degrees From the catheter shaft 204.

In some embodiments, the catheter shaft 204 is made of flexible material that may curve with the anatomy of the patient's CS 210. In certain embodiments, for example, the catheter shaft 204 may include polyether block amide, nylon, silicone, or a combination thereof. In some instances, the catheter shaft 204 may be a multi-layered and multi-material component. In some examples, the catheter shaft 204 is reinforced with a braid and can have an etched or casted liner. The braid for reinforcing the catheter shaft 204 may be made of nitinol. The liner may be made from polyetheretherketone (PEEK), polytetrafluoroethylene (PTFE), copolymers of polyamide and polyether, or a combination thereof. In some embodiments, the catheter shaft 204 is coated for lubricity with a hydrophilic coating, or other types of coating suitable for coating a catheter shaft as known by a skilled person in the art.

In some embodiments, the shunting catheter 202 has a diameter of from about 2 mm to about 5 mm. In certain embodiments, the shunting catheter 202 has a diameter of from about 2.5 mm to about 4.5 mm. In some embodiments, the shunting catheter has a diameter of from about 3 mm to about 4 mm. In certain embodiments, the shunting catheter 202 may have a diameter allowing it to pass through vessels and parts of the cardiovascular system to reach a target location.

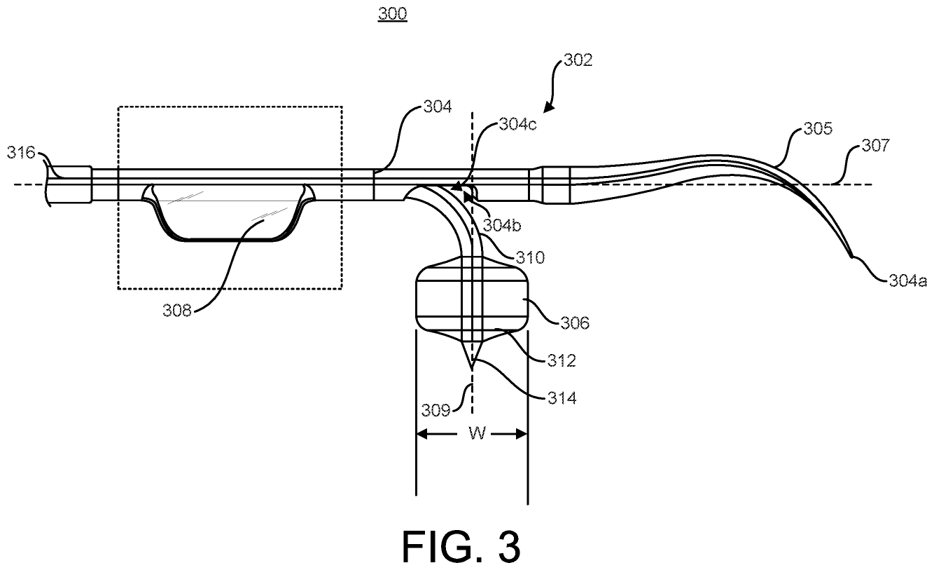

FIG. 3 is a schematic diagram of a side view of an example of a shunting device 300 and a perspective view of an apposition element 308 of the shunting device 300, in accordance with embodiments of the present disclosure. FIG. 3 is merely an example. One of the ordinary skilled in the art would recognize many variations, alternatives, and modifications. As shown, the shunting device 300 includes a shunting catheter 302 to be delivered through a patient's coronary sinus (CS). The shunting catheter 302 includes a catheter shaft 304, a shunting element 306, and an apposition element 308.

According to certain embodiments, the catheter shaft 304 has a distal end 304a, a proximal end (not shown), and a shaft lumen 304b. In some embodiments, the catheter shaft 304 is made of flexible material that may curve with the anatomy of the patient's CS. In certain embodiments, the catheter shaft 304 may include polyether block amide, nylon, silicone, and/or a combination thereof. In some instances, the catheter shaft 304 may be a multi-layered and multi-material component. In some examples, the catheter shaft 304 is reinforced with a braid and can have an etched or casted liner. The braid for reinforcing the catheter shaft 304 may be made of nitinol. The liner may be made from polyetheretherketone (PEEK), polytetrafluoroethylene (PTFE), copolymers of polyamide and polyether, or a combination thereof. In certain embodiments, the catheter shaft 304 may be injection molded or extruded. In some embodiments, the catheter shaft 304 is coated for lubricity with a hydrophilic coating, or other types of coating suitable for coating a catheter shaft as known by a skilled person in the art. In some instances, the catheter shaft 304 may have multiple lumens.

According to some embodiments, the catheter shaft 304 may include a stabilizing element such as distal tip 305 at the distal end 304a that has a curve (e.g., a pre-existing curve), for example, a curve conforming to the anatomy of a patient's CS. In some instances, the distal tip 305 may be made of a different material than other parts of the catheter shaft 304. In some instances, for example, the distal tip 305 may be made of a material more flexible than the material of other parts of the catheter shaft 304. The distal tip 305 may be injection molded or machined to have a unique geometry (e.g., a curve) for better stabilizing the catheter shaft 304 during deployment.

According to some embodiments, the distal tip 305 may have a length of from about 5 mm to about 85 mm. In certain embodiments, the catheter shaft 304 includes a shaft opening 304c. In some embodiments, a portion of the catheter shaft from the shaft opening 304c and the distal end 304a has a curve. In some embodiments, the catheter shaft 304 defines a first axis 307, and the shunting element 306 defines a second axis 309 at the second state after deployment. In certain embodiments, the second axis 309 and the first axis 307 form an angle greater than zero degree.

According to certain embodiments, the shunting element 306 is disposed in the shaft lumen 304b at a first state. In some embodiments, the shunting element 306 includes an expandable element 312 (e.g., a balloon) connected to a shunting element shaft 310 on one end, and a puncture element 314 (e.g., a needle) on the other end. In certain embodiments, the expandable element 312 is an elongated element. The shunting element 306 may be connected to the shunting element shaft 310 positioned within the shaft lumen 304b of the catheter shaft 304 at a first state (e.g., before a deployment and/or during a deployment to position the shunting element 306). In certain embodiments, the shunting element shaft 310 has a pre-determined curve. In some examples, the shunting element shaft 310 has a pre-determined curve for the shunting element 306 to deploy. In certain embodiments, the shunting element shaft is extended from the shaft lumen 304b of the catheter shaft 304 at a second state (e.g., a shunting state to use the shunting element). In some examples, the expandable element 312 may be a balloon configured to deliver ablative energy, and is expanded when the shunting element 306 is at a second state.

According to certain embodiments, the width of the expandable element 312 (w) can range from about 3 mm to about 15 mm. In some embodiments, the width of the expandable element 312 (w) can range from about 3.5 mm to about 12 mm. In certain embodiments, the width of the expandable element 312 (w) can range from about 4 mm to about 10 mm. In some embodiments, the width of the expandable element 312 (w) can range from about 4.5 mm to about 8 mm.

According to some embodiments, the shunting catheter 302 further includes an outer shaft 316 disposed outside of at least a part of the catheter shaft 304 during deployment. In some embodiments, the outer shaft 316 is made of flexible material that may curve with the anatomy of the patient's CS. In certain embodiments, for example, the outer shaft 316 may include polyether block amide, nylon, silicone, or a combination thereof. In some instances, the outer shaft 316 may be a multi-layered and multi-material component. In some examples, the outer shaft 316 is reinforced with a braid and/or can have an etched or casted liner. The braid for reinforcing the catheter shaft 304 may be made of nitinol. The liner may be made from polyetheretherketone (PEEK), polytetrafluoroethylene (PTFE), copolymers of polyamide and polyether, or a combination thereof. In certain embodiments, the outer shaft 316 may be injection molded or extruded. In some embodiments, the catheter shaft 304 is coated for lubricity with a hydrophilic coating, or other types of coating suitable for coating a catheter shaft as known by a skilled person in the art.

According to certain embodiments, the apposition element 308 is disposed within the outer shaft 316 at a first state (e.g., during deployment). In embodiments, the apposition element 308 protrudes from the catheter shaft 304 during deployment. The apposition element 308 is flexible and compressed to fit within the outer shaft 316, and configured to decompress and protrude from the catheter shaft 304 during deployment. In some embodiments, the apposition element 308 is disposed proximate to the shunting element 306 and/or the one or more shaft openings 304c. In some instances, the apposition element 308 is a braided structure including one or more nickel titanium wires. In yet some instances, the apposition element 308 is made of a flexible material having a portion protruding from the catheter shaft 304. In some examples, the flexible material may be a foam. In some instances, the flexible material may be a balloon filled with a contrast solution that shows up under fluoroscopy. In yet some instances, the flexible material may be a polymer with a radiopaque marker added for visualization. The radiopaque marker may include tantalum, gold, or any radiopaque maker known by a skilled person in the art.

In certain embodiments, the apposition element 308 is configured to appose a patient's tissue wall (e.g., the vessel wall of patient's CS) such that the shunting catheter 302 is stabilized in one position once deployed. According to some embodiments, the apposition element 308 has several benefits, one of which is the stabilization of catheter 302 after deployment. Any movement or lack thereof the protruding element (e.g., braided element 318) provides an estimated distance of how far the catheter 302 is away from a tissue wall (e.g., the vessel wall of patient's CS). In addition, in instances where the apposition element 308 includes a braided element 318, even when the element 318 is apposing a tissue wall (e.g., the vessel wall of a patient's CS), the openings between the braids still allow blood flow through the apposition element 308, thus reducing the risk of thrombus formation caused by any occlusion in the vessel.

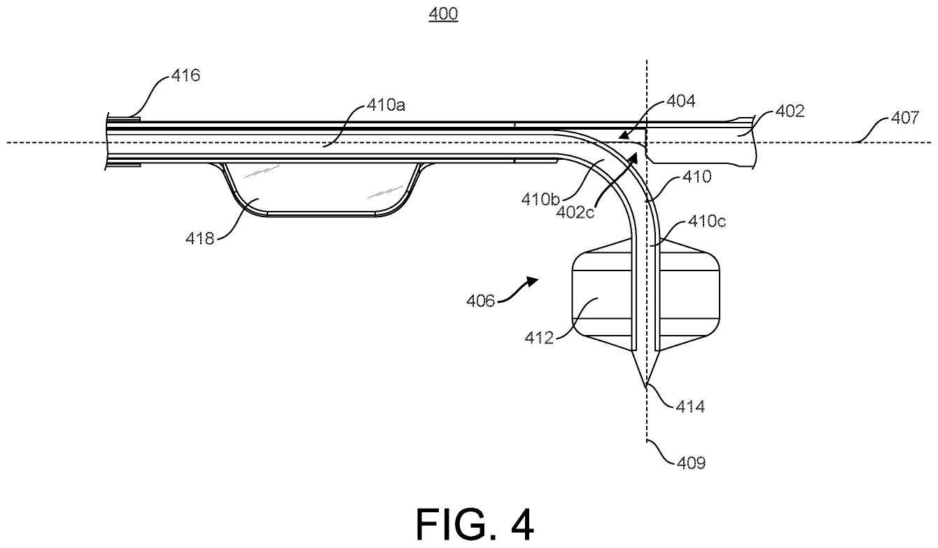

FIG. 4 is a schematic diagram of a cross-sectional view of an example of a shunting catheter 400, in accordance with embodiments of the present disclosure. As shown, the shunting catheter 400 includes a catheter shaft 402 having a shaft lumen 404, and a shunting element 406 disposed within the shaft lumen 404 at a first state (e.g., during deployment).

In some embodiments, the shunting element 406 is extended from the catheter shaft 402 at a second state. The shunting element 406 may include an expandable element 412 (e.g., a balloon) connected to a shunting element shaft 410 on one end, and a puncture element 414 (e.g., a needle) on the other end. In some examples, the expandable element 412 may be a balloon configured to deliver energy (e.g., ablative energy, radiofrequency (RF) energy, phased RF energy, thermal energy, cryogenic energy, pulse ablative energy (e.g., pulsed field ablation (PFA)), microwave energy, laser energy, ultrasound energy, etc.) to target tissue. In some embodiments, the shunting element 406 is positioned within the catheter shaft 402 at a first state (e.g., before a deployment and/or during a deployment to position the shunting element 406). In certain embodiments, the shunting element shaft 410 has a pre-determined curve. In some examples, the shunting element shaft 410 has a pre-determined curve for the shunting element 406 to deploy. In certain embodiments, the shunting element shaft 410 is extended from the catheter shaft 402 at a second state (e.g., a shunting state, a shunting state to use a shunting element).

According to some embodiments, the catheter shaft 402 includes a shaft opening 402c. In some embodiments, the catheter shaft 402 defines a first axis 407, and the shunting element 406 defines a second axis 409. In certain embodiments, the second axis 409 and the first axis 407 form an angle greater than zero degree. In certain examples, the second axis 409 and the first axis 407 forms an angle greater than 30 degrees. In some embodiments, the second axis 409 and the first axis 407 form an angle proximate to 90 degrees. In some embodiments, the second axis 409 and the first axis 407 form an angle in the range of 60 degrees to 120 degrees. In some instances, the catheter shaft 402 includes a pre-curve formed from a semi-rigid or rigid material connected to the puncture element 414. The semi-rigid or rigid material may include nitinol or stainless steel (SS) with a curve built in before deployment.

In some embodiments, the shunting element shaft 410 includes a curved portion 410b that forms an arc connecting a first straight portion of shunting element shaft 410a disposed inside the shaft lumen 404 and a second straight portion 410c of the shunting element shaft 410 extended outward from the shaft lumen 404. In embodiments, for example as shown, the curved portion 410b of the shunting element shaft 410 is adjacent the shaft opening 402c. In certain embodiments, the expandable element 412 is located at the second straight portion 410c and outside of the curved portion 410b of the shunting element shaft 410.

According to certain embodiments, the shunting catheter 400 may further include an outer shaft 416 disposed outside of the catheter shaft 402 and enclosing the catheter shaft 402, the apposition element 418 in a compressed state before deployment, and the shunting element 406. The outer shaft 416 may have a diameter of from about 8 to about 18 french, or from about 8.5 to about 16 french, or from about 9 to about 14 french, or from about 9.5 to about 12 french, or may have a diameter encompassed within these ranges. In some embodiments, for example during deployment, the outer shaft 416 is pulled back to deploy and/or position the catheter shaft 402 including the apposition element 418 and the shunting element 406.

In certain embodiments, the shunting catheter 400 includes multiple compartments (e.g., lumens) for various elements to provide more targeted control during deployment. For example, the shunting catheter 400 may include an additional lumen in between the catheter shaft 402 and the shunting element shaft 410 for more precise control during deployment of the shunting element 406. Similarly, for example, the shunting catheter 400 may include an additional lumen in between outer shaft 416 and the catheter shaft 402 for more precise control during deployment of the apposition element 418. In some embodiments, the shunting catheter 400 may include lumens for containing functional components such as a guidewire or pull wire assembly, as will be discussed further below. In yet some embodiments, the shunting catheter 400 may include additional lumens for holding shunted tissue from a tissue wall.

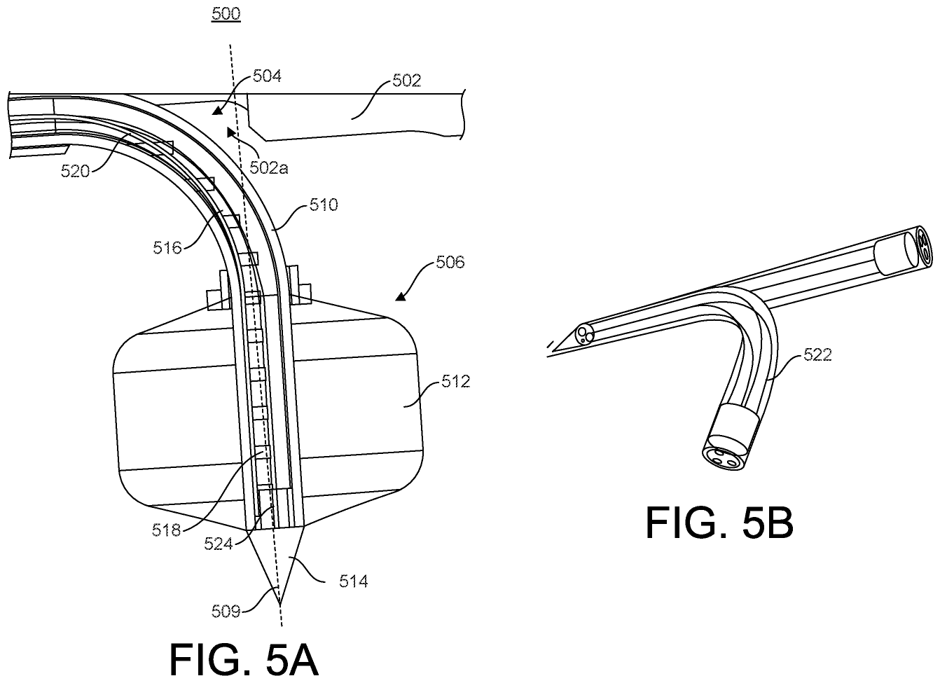

FIGS. 5A-5B are schematic diagrams of a cross-sectional view of an example of a shunting catheter 500, in accordance with embodiments of the present disclosure. As shown, the shunting catheter 500 includes a catheter shaft 502 having a shaft opening 502a, a shaft lumen 504, and a shunting element 506 disposed within the shaft lumen 504 at a first state (e.g., during deployment).

In some embodiments, the shunting element 506 is extended from the catheter shaft 502 at a second state. The shunting element 506 may include an expandable element 512 connected to a shunting element shaft 510. The shunting element shaft 510 may be further connected to a puncture element 514 (e.g., a needle). In some examples, the expandable element 512 may be a balloon configured to deliver ablative energy. In some embodiments, the shunting element 506 is positioned within the catheter shaft 502 at a first state (e.g., before a deployment and/or during a deployment to position the shunting element 506). In certain embodiments, the shunting element shaft 510 has a pre-determined curve. In some examples, the shunting element shaft 510 has a pre-determined curve for the shunting element 506 to deploy. In certain embodiments, the shunting element shaft 510 is extended from the catheter shaft 502 at a second state (e.g., a shunting state to use the shunting element 506).

In some embodiments, the shunting element 506 includes a tube 516 (e.g., a hypotube) to support the expandable element 512. The tube 516 may include a plurality of laser cuts 518 generally perpendicular to a second axis defined by the shunting element 506. In certain embodiments, the shunting element 506 includes a guidewire 520. In some instances, the tube 516 is made of stainless steel or nitinol, and may further include a pull wire assembly 522 to control the flex or angle of the puncture element 514 relative to the catheter shaft 502.

The pull wire assembly 522 may be laser welded to the tube 516 or inside the tube 516 at the distal end 524. In some embodiments, the pull wire assembly 522 may include nitinol, stainless steel (SS), cobalt, chromium, titanium, or a combination thereof. The shunting element shaft 510 may be made of a semi-rigid or rigid material to have a pre-formed angle before deployment. After the shunting element 506 is deployed, the pre-formed angle may be further adjusted using the pull wire assembly 522 to further adjust and/or stabilize the contact point between the puncture element 514 and a tissue wall (not shown).

In certain embodiments, during deployment, the guidewire 520 may be used to guide the shunting catheter 500 into the CS of a patient. In yet certain embodiments, the guidewire 520 may be used to indicate the location of the shunting catheter 500 including one or more of the components (e.g., the shunting element 506, shaft opening 502a, etc.) in the CS of a patient.

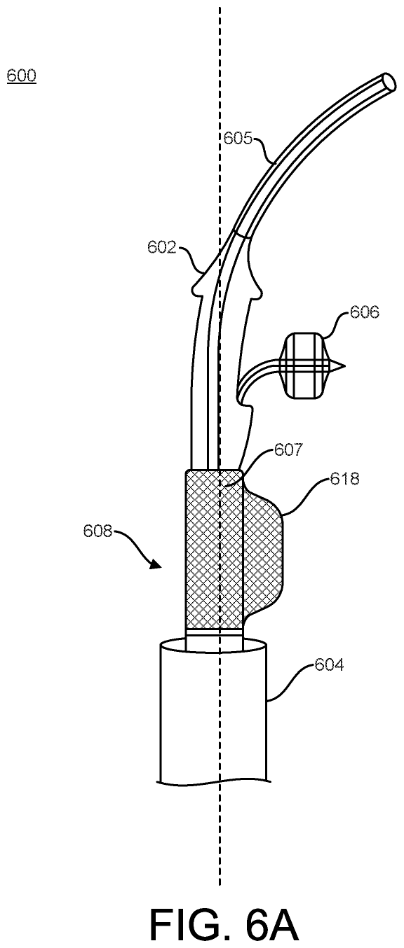

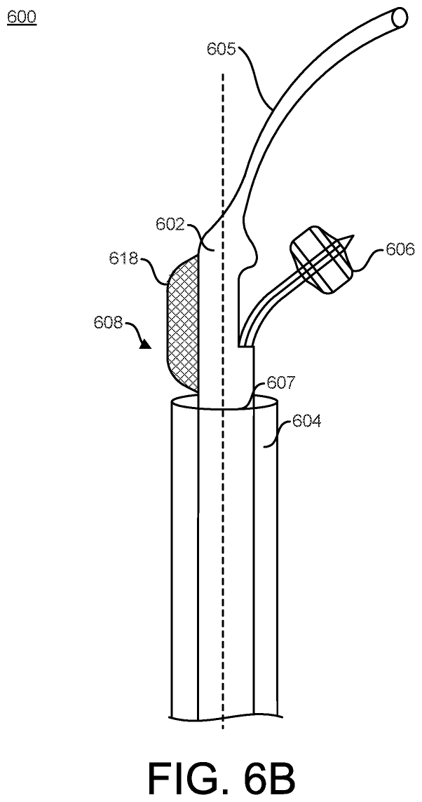

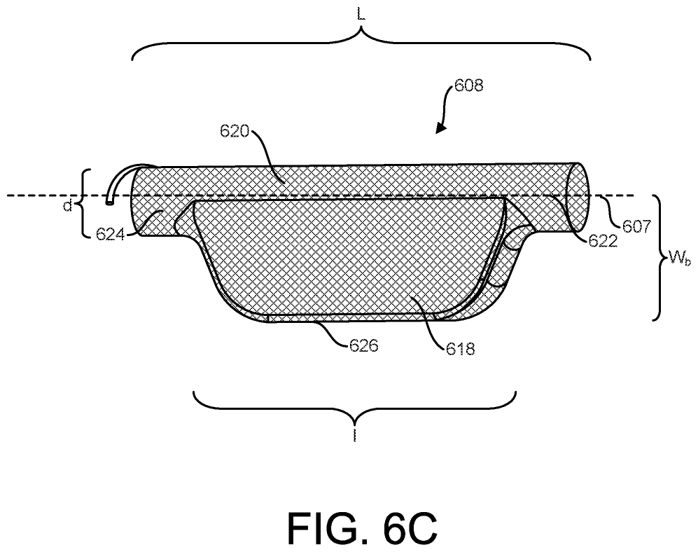

FIGS. 6A-6B are schematic diagrams of a side view of an example of a shunting catheter 600, in accordance with embodiments of the present disclosure. As shown, the shunting catheter 600 includes a catheter shaft 602 disposed within an outer shaft 604, a shunting element 606 and an apposition element 608.

According to some embodiments, the outer shaft 604 is disposed outside of at least a part of the catheter shaft 602 during deployment. The apposition element 608 is disposed within the outer shaft 604 at a first state (e.g., during deployment). In embodiments, the apposition element 608 protrudes from the catheter shaft 602 during deployment. The apposition element 608 is flexible and compressed to fit within the outer shaft 604, and configured to decompress and protrude from the catheter shaft 602 during deployment.

In some embodiments, the apposition element 608 includes a braided element 618 that is protruded from the catheter shaft 602. The braided element 618 is flexible and compressed to fit within the outer shaft 604 and configured to decompress and protrude from the catheter shaft 602 during deployment. The apposition element 608 including the braided element 618 is disposed proximate to the shunting element 606. In some embodiments, the braided element 618 includes one or more nickel titanium wires.

The apposition element 608 is configured to appose at least one wall in a patient's CS or LA such that the shunting catheter 600 is stabilized in one position once deployed. According to some embodiments, the apposition element 608 has several benefits, one of which is the stabilization of catheter 600 after deployment. Any movement or lack thereof of the protruding element (e.g., braided element 618) provides an estimated distance of how far the catheter 600 is away from a tissue wall (e.g., the vessel wall of patient's CS). In addition, in instances where the apposition element 608 includes a braided element 618, even when the element 618 is apposing a tissue wall (e.g., the vessel wall of patient's CS), the openings between the braids still allow blood flow through the apposition element 608, thus reducing the risk of thrombus formation caused by any occlusion in the vessel.

In some embodiments, for example as shown in FIG. 6A, the apposition element 608 may be located on the same side as the shunting element 606 of the first axis 607 as defined by the catheter shaft 602. In certain embodiments, for example as shown in FIG. 6B, the apposition element 608 may be located on a different side (e.g., the opposite side of the first axis 307) as the shunting element 606. The location of the apposition element 608 may vary on the catheter shaft 602 depending on a physician's need. For example, an apposition element located on the same side of the first axis 607 as the shunting element 606 may provide a better angle for the braided element 618 to have a normal or orthogonal contact with the CS wall.

According to certain embodiments, for example as shown in FIG. 6C, the apposition element 608 may include the braided element 618 and an apposition element shaft 620. In some embodiments, the apposition element shaft 620 may also include a braided material that is braided together with the braided element 618. In certain embodiments, the apposition element shaft 620 includes a distal end 622 and a proximal end 624, the distal end 622 connected or attached to the distal tip 605 of the catheter shaft 602, and the proximal end 624 connected or attached to the proximal end of the catheter shaft 602. In some examples, the apposition element shaft 620 has a length (L) longer than a length (I) of the braided element 618. In some examples, the length (L) may be from about ¼ inch to about 1 inch. In certain examples, the apposition element shaft 620 has a diameter (d) similar to the diameter of the catheter shaft 602. In some instances, the distal end 622 and proximal end 624 of the apposition element shaft 620 are attached to the catheter shaft 602 via heat shrink and/or reflow soldering.

According to some embodiments, the braided element 618 is protruding outward from the apposition element shaft 620 having a width (Wb) measured from an outer wall 626 of the braided element 618 configured to appose a CS wall to the first axis 607 defined by the catheter shaft 602. The size of the braided element 618 including its width (Wb) may vary depending on the position the apposition element 608 is in the patient's CS. Referring back to FIG. 2, the patient's coronary sinus (CS) 210 is relatively wider at the entrance CS ostium 211, and decreases in diameter as the CS 210 gets closer to the patients left and right atria. As such, the width (Wb) of the braided element 618 may also decrease as the catheter 600 travels through the patient's right atrium to conform to the shape and size of the patient's CS. In some instances, the width (Wb) may be from about 1 cm to about 2 cm.

In some embodiments, the braided element 618 may be made of a relatively rigid material to increase the pressure between the outer wall 626 of the braided element 618 and the CS wall of a patient as the shunting catheter 600 travels through the patient's CS. In some embodiments, braided element 618 may be made of a relatively soft and flexible material that deforms easily, and thus providing less pressure between the outer wall 626 of the braided element 618 and the CS wall of a patient as the shunting catheter 600 travels through the patient's CS.

In certain embodiments, the softness or rigidness of the braided element 618 may be adjusted by the material used, how tightly woven the braid is, and varying the diameter of the braids. In embodiments, for example, the braided element 618 may include nitinol braid, stainless steel (SS), cobalt, chromium, laser cut nitinol, polytetrafluoroethylene (PTFE) foam, other types of foam, polymers (e.g., shape memory polymers), polycab wires, nylon, copolymers of polyamide and polyether, polyethylene terephthalate (PET), polyurethane (PU), silicone, thermoplastic polyurethanes, polyamide, or a combination thereof. In some instances, the braided element 618 is multilayered. In some instances, the braids may be woven more tightly for a more rigid structure. In yet some instances, there may be more space in between each braid such that the braided element 618 is compressed more easily. In some examples, the braids have a diameter of about 10 micron. In some embodiments, the braids may be dip coated in ePTFE for added strength. In yet some embodiments, the braids may have electrospun ePTFE for added strength.

In some embodiments, during delivery or deployment of the catheter 600, the apposition element 608 may be used to help physician locate the catheter 600 in a cardiovascular system. For example, the width (Wb) of the braided element 618 may correlate to a point on the shunting element 606. During delivery and deployment of the catheter 600, imaging techniques such as fluoroscopy or x-ray may be used to locate the relative position of the apposition element 608 and the shunting element 606. In some instances, after deployment of the braided element 618, the location of the catheter 600 in a cardiovascular system may be estimated based on movement, or lack thereof, of the braided element 618.

In some embodiments, during delivery of the catheter 600, the braided element 618 is compressed (e.g., tied down with suture) and covered by the outer shaft 604. Once the outer shaft 604 is removed during deployment, the braided element 618 is released and self-expands to push against the cardiovascular system wall. In some instances, the shunting catheter 600 may include an additional shaft located in between the outer shaft 604 and the braided element 618 for better control of the release and decompression of the braided element 618 during deployment.

In embodiments, the apposition element 608 may be coated with filament or lamination for added strength and stability to the structure. In other words, the geometric shape of the braided element 618 may be more consistent upon deployment, and the wires making up the braids have less movement once the braided element 618 is deployed.

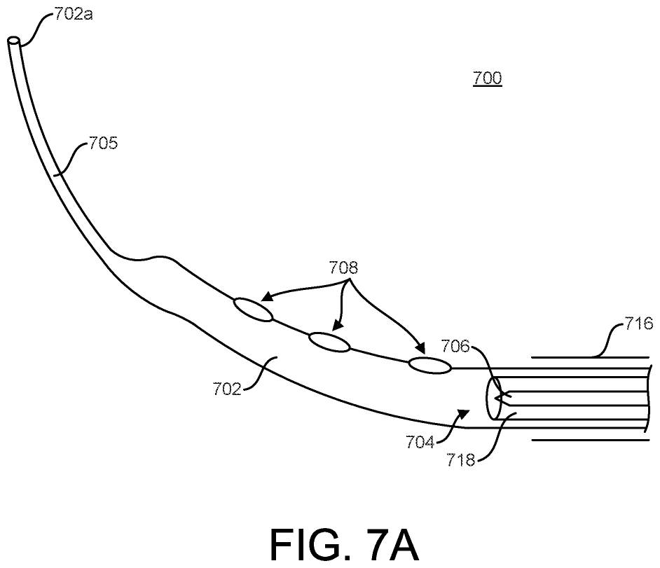

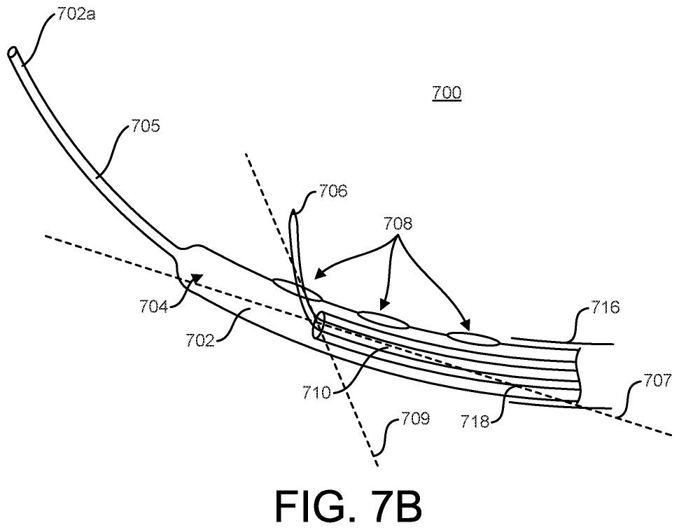

FIGS. 7A-7B are schematic diagrams of an example of a shunting

catheter 700, in accordance with embodiments of the present disclosure. As shown, the shunting catheter 700 includes a catheter shaft 702 having a shaft lumen 704, and a shunting element 706 disposed within the shaft lumen 704 at a first state (e.g., during deployment to position the shunting element 706).

According to some embodiments, the catheter shaft 702 includes a stabilizing element such as distal tip 705 at the distal end 702a that has a curve (e.g., a pre-existing curve), for example, a curve conforming to the anatomy of a patient's CS. The distal tip may have a length of from about 5 mm to about 85 mm.

According to certain embodiments, the catheter shaft 702 includes a plurality of shaft openings 708 located on a side of the catheter shaft 702, and aligned along the length of the catheter shaft 702. In some embodiments, for example as shown, the plurality of shaft openings 708 are circular in shape, and may have a diameter of from about 0.5 mm to about 4 mm. In certain embodiments, each of the plurality of shaft openings 708 has a diameter of from about 0.5 mm to about 3 mm. In some instances, the plurality of shaft openings 708 are of the same size. In yet some instances, the plurality of shaft openings 708 are of different sizes.

In certain embodiments, a shaft opening 708 may be of other shapes, for example, oval, rectangular, triangular, and/or the like. In certain embodiments, each of the plurality of shaft openings 708 are configured such that the shunting element 706 may fit through one of the plurality of shaft openings 708 during deployment. In some embodiments, the plurality of shaft openings 708 at various locations on the catheter shaft 702 provide ease for a shunt to be created at different locations along a patient's CS or LA wall.

In some embodiments, not shown in FIGS. 7A-7B, a catheter shaft may include multiple lumens for separately containing and deploying multiple shunting elements through the plurality of shaft openings 708. As such, multiple shunts may be created without having to move the catheter shaft 702 by having multiple shaft openings along a side of the catheter shaft 702. Each of the plurality of shunting elements may be separately deployed via control of each lumen that each of the shunting elements are disposed in.

In certain embodiments, the catheter shaft 702 includes 3 shaft openings, for example as shown in FIGS. 7A-7B. In certain embodiments, the catheter shaft 702 may include fewer than 3 shaft openings. In some embodiments, the catheter shaft 702 may include more than 3 shaft openings.

In some embodiments, for example as shown in FIG. 7B, the shunting element 706 is extended from the catheter shaft 702 at a second state. The shunting element 706 may be connected to a shunting element shaft 710. In some examples, the shunting element shaft 710 has a pre-determined curve for the shunting element 706 to deploy. In certain embodiments, the shunting element shaft 710 is extended from the catheter shaft 702 at a second state (e.g., a shunting state to use the shunting element 706).

According to some embodiments, the catheter shaft 702 defines a first axis 707, and the shunting element 706 defines a second axis 709. In certain embodiments, the second axis 709 and the first axis 707 forms an angle greater than zero degree. In certain examples, the second axis 709 and the first axis 707 form an angle greater than 20 degrees. In some embodiments, the second axis 709 and the first axis 707 form an angle proximate to 45 degrees. In some embodiments, the second axis 709 and the first axis 707 form an angle in the range of 60 degrees to 120 degrees. In some instances, the catheter shaft 702 includes a pre-curve formed from a semi-rigid or rigid material. The semi-rigid or rigid material may include nitinol or stainless steel (SS) with a curve built in before deployment.

The shunting catheter 700 may further include an outer shaft 716 disposed outside of the catheter shaft 702 and enclosing the catheter shaft 702. The outer shaft 716 may have a diameter of from about 8 to about 18 french, or from about 8.5 to about 16 french, or from about 9 to about 14 french, or from about 9.5 to about 12 french, or may have a diameter encompassed within these ranges. In some embodiments, for example during deployment, the outer shaft 716 is pulled back to deploy and/or position the catheter shaft 702 including the shunting element 706.

In certain embodiments, the shunting catheter 700 includes multiple compartments (e.g., lumens) for various elements to provide more targeted control during deployment. For example, the shunting catheter 700 may include an additional lumen 718 disposed in between the catheter shaft 702 and the shunting element shaft 710 for more precise control during deployment of the shunting element 706. In some embodiments, the shunting catheter 700 may include one or more additional lumens for separately containing functional components such as a guidewire or pull wire assembly (e.g., pull wire assembly 522 in FIG. 5B). In yet some embodiments, the shunting catheter 700 may include additional lumens for holding shunted tissue from a tissue wall.

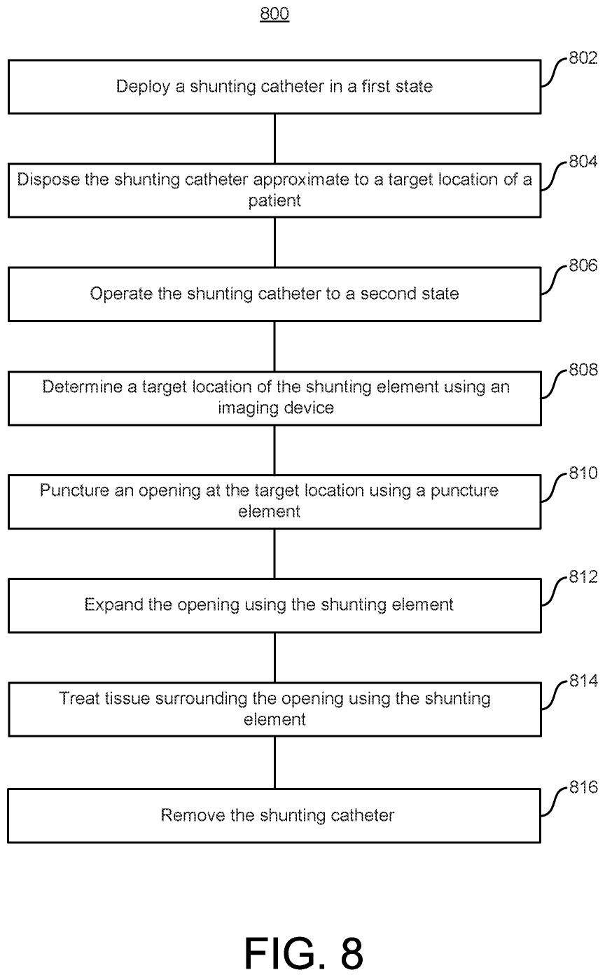

FIG. 8 is a flow diagram illustrating a process 800 of creating a shunt in a patient, in accordance with embodiments of the present disclosure. The method is described in relation to the catheters discussed previously here, however, any suitable electroporation catheter can be used in the method. Aspects of embodiments of the method may be performed, for example, by a shunting catheter system or a controller (e.g., the system 104 in FIG. 1, the controller 112 in FIG. 1). One or more steps of method are optional and/or can be modified by one or more steps of other embodiments described herein. Additionally, one or more steps of other embodiments described herein may be added to the method. In some embodiments, the shunt may be formed in a coronary sinus of a patient. In certain embodiments, the shunt includes an opening between a patient's coronary sinus and left atrium.

At step 802, the process 800 includes deploying a shunting catheter in a first state, the shunting catheter including a catheter shaft having a distal end and a proximal end and a shaft lumen, a shunting element having a proximal end and a distal end, and a puncture element disposed proximate to the distal end of the shunting element. In some embodiments, the shunting element is disposed in the shaft lumen at the first state. In certain embodiments, deploying the shunting catheter includes inserting the shunting catheter through a superior vena cava of a patient into a coronary sinus of the patient. In certain embodiments, deploying the shunting catheter includes inserting the shunting catheter through an inferior vena cava of a patient into a coronary sinus of the patient.

At step 804, the process 800 includes disposing the shunting catheter approximate to a target location of a patient. At step 806, the process 800 includes operating the shunting catheter to a second state, wherein the shunting element extends from the catheter shaft at an angle greater than zero degree at the proximal end of the shunting element at the second state. In some embodiments, the shunting catheter includes an apposition element disposed proximate to the shunting element, and the apposition element is protruded from the catheter shaft at the second state.

At step 808, the process 800 may include determining a target location of the shunting element using an imaging device. In some embodiments, the imaging device includes one or more visualization elements disposed proximate the shunting element.

At step 810, the process 800 includes puncturing, using the puncture element, an opening at the target location. In some embodiments, the target location is at a coronary sinus of a patient.

At step 812, the process 800 includes expanding the opening using the shunting element. In certain embodiments, the catheter shaft has a shaft opening, and the shunting element extends from the catheter shaft through the shaft opening.

At step 814, the process 800 may include treating tissue (e.g., by ablation) surrounding the opening using the shunting element. In some embodiments, for example, the shunting element includes an expandable element (e.g., a balloon) that is expanded at the second state and configured to deliver energy (e.g., ablative energy, radiofrequency (RF) energy, phased RF energy, thermal energy, cryogenic energy, pulse ablative energy (e.g., pulsed field ablation (PFA)), microwave energy, laser energy, ultrasound energy, etc.) to the tissue surrounding the opening.

At step 816, the process 800 may include removing the shunting catheter from a patient. In some embodiments, the process 800 may include removing the shunting catheter, which includes removing the catheter shaft, the puncture element, and the shunting element. In certain embodiments, the process 800 does not leave any implant device at the target location. In some embodiments, a shunt is formed by creating an opening between a coronary sinus and a left atrium of a patient. In certain embodiments, the shunting catheter is removed from the coronary sinus of the patient. In certain embodiments, the formed shunt is an opening that does not include an implant (e.g., a frame or structure to support an opening). In some embodiments, the shunt consists of an opening between the coronary sinus and the left atrium of a patient; where the shunt does not include an implant.

According to some embodiments, the process 800 includes generating a shunt using a shunting element of a shunting catheter. In certain embodiments, the shunt includes an expanded opening between the coronary sinus and left atrium of a patient. In some embodiments, the shunt does not include any implant.

According to some embodiments of the present disclosure, a shunting catheter includes: a catheter shaft having a distal end and a proximal end, the catheter shaft including a shaft lumen; a shunting element disposed in the shaft lumen at a first state and extended from the catheter shaft at a second state; and an apposition element disposed proximate to the shunting element, the apposition element being protruded from the catheter shaft at the second state.

In certain embodiments, the catheter shaft defines a first axis; wherein the shunting element defines a second axis at the second state; wherein the second axis and the first axis form an angle greater than zero degree. In some embodiments, the shunting element includes an expandable element that is expanded at the second state. In certain embodiments, the shunting element includes a tube to support the expandable element. In some embodiments, the tube includes a plurality of cuts generally perpendicular to the second axis.

In some embodiments, the catheter shaft has a shaft opening, wherein the shunting element extends from the catheter shaft through the shaft opening. In certain embodiments, the shaft opening is not at the distal end of the catheter shaft. In some embodiments, a portion of the catheter shaft between the shaft opening and the distal end has a curve. In some embodiments, the shunting element includes a guidewire. In certain embodiments, the shunting element includes a puncture element.

In certain embodiments, the shunting catheter further includes: an outer shaft disposed outside of at least a part of the catheter shaft; wherein the apposition element is disposed within the outer shaft. In some embodiments, the apposition element includes a braided element protruded from the catheter shaft at the second state. In certain embodiments, the braided element includes one or more nickel titanium wires.

According to some embodiments, a shunting catheter includes a catheter shaft having a distal end and a proximal end, the catheter shaft including a shaft lumen; a shunting element disposed in the shaft lumen at a first state and extended from the catheter shaft at a second state; wherein the catheter shaft defines a first axis; wherein the shunting element defines a second axis at the second state; wherein the second axis and the first axis form an angle greater than zero degree. In some embodiments, the distal end of the catheter shaft has a curve.

In certain embodiments, the shunting catheter further includes an apposition element disposed proximate to the shunting element, the apposition element being protruded from the catheter shaft at the second state.

According to some embodiments, a shunting catheter system includes a shunting catheter including: a catheter shaft having a distal end and a proximal end, the catheter shaft including a shaft lumen; a shunting element disposed in the shaft lumen at a first state and extended from the catheter shaft at a second state; and an apposition element disposed proximate to the shunting element, the apposition element being protruded from the catheter shaft at the second state. In some embodiments, the shunting catheter system further includes an energy source connected to the shunting catheter; and a controller connected to the energy source including one or more processors; wherein the one or more processors are configured to control the energy source to deliver energy to the shunting catheter.

In certain embodiments, the shunting catheter system further includes an imaging device including: one or more visualization elements disposed proximate the shunting element for determining a location of the shunting element within a heart of a patient, and a display for visualizing the location.

According to certain embodiments, a method for creating a shunt, includes deploying a shunting catheter in a first state, the shunting catheter including: a catheter shaft having a distal end and a proximal end, the catheter shaft including a shaft lumen; a shunting element having a proximal end and a distal end, wherein the shunting element is disposed in the shaft lumen at the first state; and a puncture element disposed proximate to the distal end of the shunting element; disposing the shunting catheter approximate to a target location of a patient; operating the shunting catheter to a second state, wherein the shunting element extends from the catheter shaft at an angle greater than zero degree at the proximal end of the shunting element at the second state; puncturing, using the puncture element, an opening at the target location; and expanding the opening using the shunting element.

In some embodiments, the shunting catheter further includes an apposition element disposed proximate to the shunting element, wherein the apposition element is protruded from the catheter shaft at the second state. In certain embodiments, the catheter shaft has a shaft opening, wherein the shunting element extends from the catheter shaft through the shaft opening.

In some embodiments, the method further includes determining a location of the shunting element using an imaging device. In certain embodiments, the imaging device includes one or more visualization elements disposed proximate the shunting element. In some embodiments, the shunting element includes an expandable element that is expanded at the second state.

In certain embodiments, the method further includes treating tissue surrounding the opening using the shunting element. In some embodiments, the target location is at a coronary sinus of the patient. In certain embodiments, deploying the shunting catheter in the first state includes inserting the shunting catheter through a superior vena cava or an inferior vena cava of the patient into a coronary sinus of the patient.

In some embodiments, the method further includes removing the shunting catheter from the patient. In certain embodiments, the method further includes generating the shunt using the shunting element; wherein the shunt includes an expanded opening between the coronary sinus and a left atrium of the patient. In some embodiments, the shunt does not include any implant.

Various modifications and additions can be made to the exemplary embodiments discussed without departing from the scope of the present disclosure. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. A shunting catheter, comprising:
a catheter shaft having a distal end and a proximal end, the catheter shaft including a shaft lumen;
a shunting element disposed in the shaft lumen at a first state and extended from the catheter shaft at a second state; and
an apposition element disposed proximate to the shunting element, the apposition element being protruded from the catheter shaft at the second state.

2. The shunting catheter of claim 1, wherein the catheter shaft defines a first axis;
wherein the shunting element defines a second axis at the second state; wherein the second axis and the first axis form an angle greater than zero degree.

3. The shunting catheter of claim 2, wherein the shunting element includes an expandable element that is expanded at the second state.

4. The shunting catheter of claim 3, wherein the shunting element includes a tube to support the expandable element.

5. The shunting catheter of claim 4, wherein the tube includes a plurality of cuts generally perpendicular to the second axis.

6. The shunting catheter of claim 1, wherein the catheter shaft has a shaft opening, wherein the shunting element extends from the catheter shaft through the shaft opening.

7. The shunting catheter of claim 6, wherein the shaft opening is not at the distal end of the catheter shaft.

8. The shunting catheter of claim 6, wherein a portion of the catheter shaft between the shaft opening and the distal end has a curve.

9. The shunting catheter of claim 1, wherein the shunting element includes a guidewire.

10. The shunting catheter of claim 1, wherein the shunting element includes a puncture element.

11. The shunting catheter of claim 1, further comprising an outer shaft disposed outside of at least a part of the catheter shaft;
wherein the apposition element is disposed within the outer shaft.

12. The shunting catheter of claim 1, wherein the apposition element includes a braided element protruded from the catheter shaft at the second state.

13. The shunting catheter of claim 12, wherein the braided element includes one or more nickel titanium wires.

14. A shunting catheter, comprising:
a catheter shaft having a distal end and a proximal end, the catheter shaft including a shaft lumen;
a shunting element disposed in the shaft lumen at a first state and extended from the catheter shaft at a second state;
wherein the catheter shaft defines a first axis;
wherein the shunting element defines a second axis at the second state;
wherein the second axis and the first axis form an angle greater than zero degree.

15. The shunting catheter of claim 14, wherein the distal end of the catheter shaft has a curve.

16. The shunting catheter of claim 14, further comprising:
an apposition element disposed proximate to the shunting element, the apposition element being protruded from the catheter shaft at the second state.

17. A shunting catheter system, comprising:
a shunting catheter comprising:
a catheter shaft having a distal end and a proximal end, the catheter shaft including a shaft lumen;
a shunting element disposed in the shaft lumen at a first state and extended from the catheter shaft at a second state; and
an apposition element disposed proximate to the shunting element, the apposition element being protruded from the catheter shaft at the second state; and
an energy source connected to the shunting catheter; and
a controller connected to the energy source comprising one or more processors;
wherein the one or more processors are configured to control the energy source to deliver energy to the shunting catheter.

18. The shunting catheter system of claim 17, further comprising:
an imaging device comprising:
one or more visualization elements disposed proximate the shunting element for determining a location of the shunting element within a heart of a patient, and
a display for visualizing the location.

19. A method for creating a shunt, comprising:
deploying a shunting catheter in a first state, the shunting catheter comprising:
a catheter shaft having a distal end and a proximal end, the catheter shaft including a shaft lumen;
a shunting element having a proximal end and a distal end, wherein the shunting element is disposed in the shaft lumen at the first state; and
a puncture element disposed proximate to the distal end of the shunting element;
disposing the shunting catheter approximate to a target location of a patient;
operating the shunting catheter to a second state, wherein the shunting element extends from the catheter shaft at an angle greater than zero degree at the proximal end of the shunting element at the second state;
puncturing, using the puncture element, an opening at the target location; and
expanding the opening using the shunting element.

20. The method of claim 19, wherein the shunting catheter further comprises an apposition element disposed proximate to the shunting element, wherein the apposition element is protruded from the catheter shaft at the second state.

21. The method of claim 19, wherein the catheter shaft has a shaft opening, wherein the shunting element extends from the catheter shaft through the shaft opening.

22. The method of claim 19, further comprising determining a location of the shunting element using an imaging device.

23. The method of claim 22, wherein the imaging device comprises one or more visualization elements disposed proximate the shunting element.

24. The method of claim 19, wherein the shunting element includes an expandable element that is expanded at the second state.

25. The method of claim 24, further comprising:
treating tissue surrounding the opening using the shunting element.

26. The method of claim 19, wherein the target location is at a coronary sinus of the patient.

27. The method of claim 19, wherein deploying the shunting catheter in the first state comprises inserting the shunting catheter through a superior vena cava or an inferior vena cava of the patient into a coronary sinus of the patient.

28. The method of claim 27, further comprising:
removing the shunting catheter from the patient.

29. The method of claim 28, further comprising:
generating the shunt using the shunting element;
wherein the shunt includes an expanded opening between the coronary sinus and a left atrium of the patient.

30. The method of claim 29, wherein the shunt does not include any implant.
